(19) United States
(12) Patent Application Publication
(10) Pub. No.: US 2004/0191345 A1
(43) Pub. Date: Sep. 30, 2004

(54) New formulations and use thereof

(76) Inventors: Fredrik Nicklasson, Helsingborg (SE); Kristina Thyresson, Helsingborg (SE); Nils-Olof Lindberg, Helsingborg (SE); Alice C. Martino, Kalamazoo, MI (US); Katarina Lindell, Helsingborg (SE)

Correspondence Address: Evan J. Federman, Legal Division, Warner-Lambert Company, LLC, 201 Tabor Road, Morris Plains, NJ, 07950, US

(21) Appl. No.: 10/816,673

(22) Filed: Apr. 2, 2004

Related U.S. Application Data
(60) Provisional application No. 60/460,214, filed on Apr. 3, 2003.

(30) Foreign Application Priority Data
Mar. 26, 2003 (SE) 0300830-7

(51) Int. Cl. A61K 31/137; A61K 35/78

(52) U.S. Cl. 424/776; 514/651

(57) ABSTRACT

A tolterodine-containing pharmaceutical formulation that comprises cocoa powder, process for manufacturing the formulation and use of the formulation for treating overactive bladder.

This application hereby claims priority from U.S. provisional application No. 60/460,214, filed Apr. 3, 2003.

### FIELD OF THE INVENTION

This invention relates to novel orally administered pharmaceutical formulations of tolterodine, optionally comprising salts, complexes, prodrugs and metabolites thereof, to the use of tolterodine, optionally comprising salts, prodrugs and metabolites thereof, for the manufacturing of a medicament to be administered orally for achieving an effect against overactive bladder, and to methods of treating overactive bladder by oral administration of tolterodine, optionally comprising salts, prodrugs and metabolites thereof.

### BACKGROUND

Tolterodine is an effective and safe compound for treatment of overactive bladder. The synthesis of tolterodine and its utility for the treatment of overactive bladder is disclosed in U.S. Pat. No. 5,382,600 (Pharmacia & Upjohn A B). An optimal efficacy/side effect profile is obtained at an oral dosage of 1 or 2 mg twice daily.

Tolterodine has a molecular weight of 325.0 and 475.6 as the tartrate salt. The enantiomeric purity is >99%. The pKₐ value is 9.87 and the solubility in water is about 11 mg/ml at room temperature. The partition coefficient (Log P) between n-octanol and phosphate buffer at pH 7.32 is 1.83.

### Tolterodine, PNU-200583

### N,N-diiso-propyl-3-(2-hydroxy-5-methylphenyl)-3-phenylpropanamine

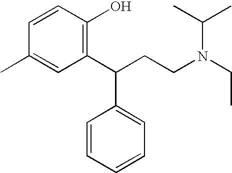

The major metabolic pathway for the metabolism of tolterodine is mediated by cytochrome P450 2D6 leading to the formation of a 5-HM metabolite, (R)—N,N-diisopropyl-3-(2-hydroxy-5-hydroxymethylphenyl)-3-phenylpropanamine. This metabolite has a similar pharmacological profile as tolterodine—see Nilvebrant L, Gillberg P-G, Sparf B. “Antimuscarinic potency and bladder selectivity of PNU-200577, a major metabolite of tolterodine.” Pharmacol. Toxicol. (1997) 81:195-207. For the similarity to tolterodine in pharmacological profile, see Brynne N, Dalén P, Alván G, Bertilsson L and Gabrielsson J, Clin Pharmacol Ther 1998 (63):529-39.

A N-dealkylated metabolite is mediated by CYP3A and may be further metabolized to a N-dealkylated 5-hydroxymetabolite. See the below scheme.

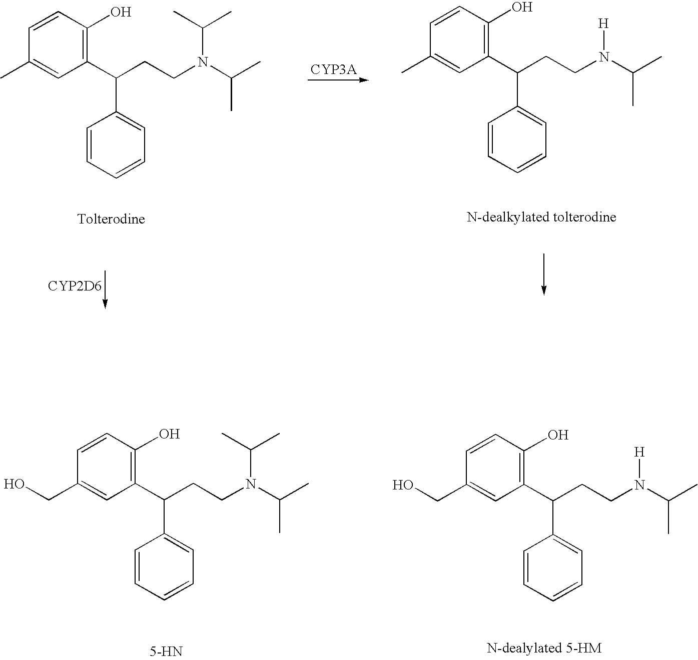

One further metabolite is formed when a carboxylic acid group is formed at the CH₂OH group of the 5-HM metabolite. Still one further metabolite is formed when a carboxylic acid group is formed at the CH₂OH group of the N-dealkylated 5-HM metabolite.

Tolterodine of the present invention encompasses the R-isomer, the S-isomer and the racemic mixture as well as salts, complexes, prodrugs and metabolites thereof. Major effects are obtained from the R-isomer and the racemic mixture as well as from salts, complexes, prodrugs and metabolites thereof. Examples of salts are tolterodine 1-tartrate and tolterodine mesylate. Examples of complexes are complexes between tolterodine and beta-cyclodextrine and tolterodine and ion exchange compositions, such as ion exchange resinates.

### PRIOR ART

Above-mentioned U.S. Pat. No. 5,382,600 does not disclose any formulation similar to the ones of the present invention.

WO 98/03067 discloses transdermal administration of the S-isomer of tolterodine.

WO 00/12070 discloses transdermal administration of the R-isomer and of the racemate of tolterodine.

No prior art on tolterodine-containing formulations similar to the ones of the present invention has been found.

Chocolate, which is different from cocoa powder as such, is very rarely used as an ingredient in pharmaceutical products, hitherto only in laxatives. One example is Ex-Lax® being chocolated laxative pieces marketed by Novartis comprising sennosides. Purex, a laxative wherein phenolphthalein was formulated with chocolate, was marketed in the 1950s. It is not known any compositions comprising tolterodine and chocolate.

It has now surprisingly been found that an orally administered pharmaceutical formulation of tolterodine, which may be administered without liquid, having sufficient taste masking of badly tasting ingredients, such as tolterodine and optional buffering agents, is obtained by tolterodine-containing formulations comprising cocoa powder as taste masker and texturizer. No similar formulations have been disclosed hitherto and the skilled person would not without inventive efforts have conceived the formulations of the present invention.

Hence the present invention, as further described below, is both new and inventive.

### SUMMARY OF THE INVENTION

The present invention provides an orally administered pharmaceutical formulation of tolterodine, optionally comprising salts, prodrugs and metabolites thereof for achieving an effect against overactive bladder, comprising detrusor instability, detrusor hyperreflexia, urinary frequency, urinary urgency and urge incontinence. The administration can be to a human being or to an animal.

The administration may be accomplished without the addition of liquid. Administration without added liquid is a big advantage in all those situations where e g clean water or other suitable liquid is not available, such as on travel. Also the administration is discreet being a big advantage e g at lectures and on the theatre. Further, use of the present formulation, which should melt in the mouth rather than be swallowed, is of a great advantage to all those persons having difficulties in swallowing a traditional tablet. A particularly useful dosage form of the present invention is thus a formulation that disintegrates or melts in the mouth without need for drinking water or other fluid.

The formulation is a dosage form comprising a therapeutically effective amount of tolterodine. A “therapeutically effective amount” herein is an amount sufficient to achieve an effect against overactive bladder, comprising detrusor instability, detrusor hyperreflexia, urinary frequency, urinary urgency and urge incontinence. By “urinary frequency” is primarily meant a need to urinate more than 8 times over 24 hours or more than 2 times per night. By “urinary urgency” is primarily meant frequent, strong and sudden needs to urinate. By “urge incontinence” is primarily meant involuntary urination after a sudden need to urinate.

It is preferred that the amount of tolterodine be lower than an amount causing significant side effects.

The invention is adapted for discreet self-administration. By “discreet self-administration” herein is meant self-administration that does not draw attention to the existence of a need for therapy.

Also provided by the present invention are methods of use of formulations of the present invention for treatment of overactive bladder, and a method of use of a formulation of the invention for preparing a medicament. Other features of this invention will be in part apparent and in part pointed out hereinafter.

An object of the invention is to provide novel orally administered pharmaceutical formulations of tolterodine comprising cocoa powder.

A second object of the invention is to provide methods for preparing said formulations.

A third object of the invention is methods for using said formulations therapy for treating overactive bladder.

Further objects of the invention will become apparent to one skilled in the art, and still other objects will become apparent hereinafter from the specification and claims.

The main advantages provided by a formulation according to the present invention are:

1) The formulation provides for adequate taste masking;

2) The formulation does not require any added liquid at the time of administration;

3) By not adding liquid at administration use of the formulation does not increase the need for therapy, as there is no increase in the urinary burden—in comparison with conventional tablets being administered together with liquid.

4) The formulation provides for discreet self-administration;

5) The formulation does not give an immediate patient-perceived association with medicines, as do traditional tablets.

6) The formulation may provide for rapid transmucosal absorption, especially when buffering agents are added.

### DETAILED DESCRIPTION OF THE INVENTION

It is the primary object of the present invention to provide pharmaceutical tolterodine-containing formulations useful for treatment of overactive bladder, comprising detrusor instability, detrusor hyperreflexia, urinary frequency, urinary urgency and urge incontinence.

More specifically it is the object of the invention to provide such a tolterodine-containing formulation, for transmucousal delivery, that mainly disintegrates and/or melts in the oral cavity with or without the aid of salivary fluid or mechanical erosion, or a combination thereof, after which the formulation may show adhesiveness towards tissues in the oral cavity.

Preferably the formulation is such that it does not require addition of liquid at the time of administration. By not adding liquid at administration use of the formulation does not increase the need for therapy, as there is no increase in the urinary burden—in comparison with conventional tablets being administered together with liquid. No additional urine is produced.

Optional addition of buffering agents provides for a transient change in local pH of the saliva. Thereby a higher fraction of tolterodine is transformed into its less ionized form. Thereupon the transmucousal permeation is facilitated, which enhances the absorption of the active agent. For those skilled in the art it is evident that the choice of the buffering system is dependent on the one or more pKₐs of the active agent.

It has surprisingly been found that a sufficient taste masking of badly tasting ingredients, such as tolterodine itself and/or buffering agents, is achieved through the use of cocoa powder. The cocoa powder acts as taste masker and texturizer.

Cocoa powder is defined as cocoa nib with some fat removed and ground into a powder. Cocoa nib is defined as cocoa beans with the shell removed. Cocoa butter is defined as fat expelled from the center (kernels or nib) of cocoa beans.

Cocoa powder is prepared from roasted cocoa beans. It is a complex compound, which consists of starch, cocoa butter, amino acids, proteins, xanthines, amines, mono- and polysaccharides, phospholipids, flavonoids, pyrazines, etc.

A preferred embodiment is a formulation, weighing around 400 mg, having the following preferred formulation (w/w):

| Ingredient | Amount (%) | Function |
|---|---|---|
| Tolterodine 1-tartrate | 0.25 | Active |
| Hydrogenated soybean oil | 43.55 | Lipid ingredient |
| Cocoa powder | 18.00 | Taste masker/texturizer |
| Mannitol | 18.00 | Diluent |
| Maize starch | 13.35 | Diluent |
| Aspartame | 0.15 | Sweetener |
| Acesulfame-K | 0.10 | Sweetener |
| Titanium dioxide | 2.00 | Coloring agent |
| Monosodium glutamate | 0.60 | Taste modifier |
| Mint and vanilla flavors | 3.00 | Flavoring agents |
| Soy lecithin | 1.00 | Emulsifier |

### EXAMPLES

below follows non-limiting examples on preparation of embodiments of the present invention.

### Example 1

### Preparation of a Preferred Embodiment

A formulation, weighing around 400 mg, is prepared with the following preferred composition (w/w):

| Ingredient | Amount (%) | Function |
|---|---|---|
| Tolterodine 1-tartrate | 0.25 | Active |
| Hydrogenated soybean oil | 43.55 | Lipid ingredient |
| Cocoa powder | 18.00 | Taste masker/texturizer |
| Mannitol | 18.00 | Diluent |
| Maize starch | 13.35 | Diluent |
| Aspartame | 0.15 | Sweetener |
| Acesulfame-K | 0.10 | Sweetener |
| Titanium dioxide | 2.00 | Coloring agent |
| Monosodium glutamate | 0.60 | Taste modifier |
| Mint and vanilla flavors | 3.00 | Flavoring agents |
| Soy lecithin | 1.00 | Emulsifier |

Cocoa powder may be used in a non-alkalized form and in an alkalized form. Both are useful in the present formulations. Alkalized cocoa powder is preferred when a somewhat milder taste is desirable.

A part of the hydrogenated soybean oil is melted. The solid components, i e tolterodine 1-tartrate, cocoa powder, mannitol, maize starch, aspartame, acesulfame-K, titanium dioxide, monosodium glutamate and the flavoring agents if solid, are added and mixed. A reduction of particle size of the solid components is performed by milling in a roll-refiner. If the solid components have already got the required particle size, e g by milling before the mixing with the fatty components, roll refining is dispensed with. After treatment in the roll-refiner the mixture is mixed with the rest of the melted fatty components or remelted, if solidified, and mixed with the rest of the melted hydrogenated soybean oil. A mixing of the melt is performed in a suitable mixer. The liquid components, i e soy lecithin and the flavoring agents if liquid, are added. Tablets or other solid dosage forms are subsequently made using suitable techniques, such as molding, extrusion or congealing, including pastillation, when necessary after suitable preconditioning. Also other suitable manufacturing methods may be used.

### Example 2

### Preparation of Another Embodiment

In essentially the same way as in Example 1 is manufactured a formulation, weighing around 500 mg, having the following preferred composition (w/w):

| Ingredient | Amount (%) | Function |
|---|---|---|
| Tolterodine 1-tartrate | 0.20 | Active |
| Cocoa powder | 50.00 | Taste masker/texturizer |
| Hydrogenated soybean oil | 44.00 | Lipid ingredient |
| Titanium dioxide | 2.50 | Coloring agent |
| Sodium chloride | 0.55 | Taste modifier |
| Aspartame | 0.15 | Sweetener |
| Acesulfame-K | 0.10 | Sweetener |
| Vanilla flavour | 1.50 | Flavoring agent |
| Soy lecithin | 1.00 | Emulsifier |

### Example 3

### Preparation of Further Embodiments

In essentially the same way as in Example 1 are manufactured formulations with a weight from around 200 mg to around 1000 mg having the below composition (w/w):

| | Amount | |
|---|---|---|
| Ingredient | (%) | Function |
| Tolterodine | 0.1-2 | Active |
| (base, prodrug, metabolite, salt or complex) | | |
| Cocoa butter equivalents (CBEs) | 35-55 | Lipid |
| | | ingredient |
| Cocoa powder | 8-55 | Taste masker/ |
| | | texturizer |
| Water-soluble or dispersible diluents, | 0-40 | Diluent |
| preferably as fine particulate powder | | |
| Sweetening agents | 0.2-3 | Sweetener |
| Buffering agents | 0-10 | Buffer |
| Flavoring agents | 0-4 | Flavor |
| Bitterness modifying agents | 0-3 | Taste modifier |
| Emulsifier/solubilizer | 0.3-6 | Emulsifier |
| Coloring agent | 0-3 | Coloring |
| | | agent |

### Example 4

### Preparation of Alternative Embodiments

Useful embodiments are obtained by exchanging some of the excipients in the embodiments of the above examples for equivalently functioning alternative compounds.

The cocoa powder may be used in its non-alkalized form, its alkalized form or in a mixture thereof.

The diluents may be selected from one or more of the compounds sucrose, fructose, glucose, galactose, lactose, maltose, invert sugar, a pharmaceutically acceptable polyol such as xylitol, sorbitol, maltitol, mannitol, isomalt and glycerol, or polydextrose, or starch, or any mixture thereof, but only to such an extent that the taste-masking effect of the cocoa-powder remains sufficient.

The lipid ingredient, being fatty components, may be chosen from one or more of the following compounds:

cocoa butter and cocoa butter alternatives, including cocoa butter equivalents (CBE), cocoa butter substitutes (CBS), cocoa butter replacers (CBR) and cocoa butter improvers (CBI),

coconut, palmkernel oil and other similar oils characterized by being predominantly based on lauric and myristic acids,

palm oil, shea butter, karite butter, illipe butter, mango kernel oil, sal fat and other similar fats characterized by being predominantly based on palmitic, oleic and stearic acids,

corn oil, sunflower oil, hybrid sunflower oil, soybean oil, rapeseed oil, canola oil, olive oil, ricebran oil, cottonseed oil, arachis (peanut, groundnut) oil and other oils characterized by being predominantly based on oleic, linoleic and linolenic acids and hydrogenated to a suitable melting point,

fish oil, tallow, lard, butterfat and other animal derived fats, and

synthetic fats, reesterified fats, hard fats obtained by a chemical reaction of fatty acids with glycerol using no, acidic, alkaline or enzymatic catalysis,

whereby said compound/s is/are used as a single component or mixed with each other, being either crude or refined using physical or alkaline refining, or being subjected to further processing including catalytic hydrogenation, interesterification, transesterification and fractionation.

The optional buffering agent/s may be selected from one or more of carbonates, bicarbonates, acetates, gluconates, glycerophosphates, phosphates or glycinates of sodium, potassium or ammonium, or mixtures thereof. Most phosphates are though less suitable because their taste usually is disagreeable and difficult to mask. Addition of buffering agents/s may increase the uptake through the buccal mucosa.

The sweetener may selected from one or more artificial sweeteners, such as sucrose, aspartame, acesulfame potassium, saccharine, sodium saccharine, cyclamate, glycyrrhizine, thaumatin (talin), sucralose, dihydrochalcone (neohesperidin dihydrochalcone), alitame, miraculin (miracle fruit), monellin (serendipity berry), stevside and/or salts thereof.

The emulsifier is preferably soy lecithin and/or egg lecithin, but may be exchanged for

a nonionic surfactant, such as poloxamer, polyoxyethylene alkyl ether, polyoxyethylene castor oil derivative, polyoxyethylene sorbitan fatty acid ester, monoglyceride, diglyceride and esther thereof, polyoxyethylene stearate, polyglycerolester of fatty acids, including polyglycerolpolyricinoleic acid (PGPR), sorbitan fatty acid ester,

an anionic surfactant, such as fatty acid, soap of fatty acid, lactylate, especially sodium and/or calcium stearoyllactylate, sodium lauryl sulfate and latanol,

a zwitterionic surfactant, such as zwitterionic phospholipid, such as phosphatidylcholine and phosphatidylethanolamine,

or mixtures, fractions or derivatives thereof or with lecithin.

Formulations according to the present inventions primarily constitute meltable and/or suckable oral tablets, but also include other suitable dosage forms for oral administration such as buccal patches, buccal paste and buccal sprays.

The present invention also encompasses tolterodine-containing formulations further comprising one or more other active agents having an effect against overactive bladder, such as oxobutynin, emepromium, trospium, propanetheline and darifenacin.

Further, the present invention encompasses treating overactive bladder in a subject through administration to a subject of a tolterodine-containing orally administered pharmaceutical formulation as presented above, optionally together with one or more other agents having an effect against overactive bladder, and further optionally concomitantly with administration of agents for treating overactive bladder through one or more other routes of administration, such as through transdermal administration, peroral administration, administration by inhalation, administration by creams, salves and vagitories, and/or administration by injection.

1. An orally administered pharmaceutical formulation comprising tolterodine, and cocoa powder.

2. The formulation according to claim 1, wherein tolterodine comprises its R-isomeric form.

3. The formulation according to claim 1, wherein tolterodine comprises its S-isomeric form.

4. The formulation according to claim 1, wherein tolterodine comprises its racemic form.

5. The formulation according to claim 1, further comprising the tolterodine metabolite (R)—N,N-diisopropyl-3-(2-hydroxy-5-hydroxymethylphenyl)-3-phenylpropanamine.

6. The formulation according to claim 1 further comprising at least one lipid.

7. The formulation according to claim 6, wherein said lipid is selected from
cocoa butter, cocoa butter equivalents, cocoa butter substitutes, cocoa butter replacers and cocoa butter improvers, coconut oil, palmkernel oil, palm oil, shea butter, karite butter, illipe butter, mango kernel oil, sal fat, corn oil, sunflower oil, hybrid sunflower oil, soybean oil, rapeseed oil, olive oil, ricebran oil, cottonseed oil, arachis (peanut, groundnut) oil, fish oil, tallow, lard, butterfat and mixtures thereof.

8. The formulation according to claim 7, wherein said lipid is selected from the group consisting of cocoa butter equivalents, cocoa butter substitutes and cocoa butter replacers.

9. The formulation according to claim 1 further comprising at least one buffering agent.

10. The formulation according to claim 9, wherein said buffering agent is selected from the group consisting of carbonates, bicarbonates, acetates, gluconates, glycerophosphates, phosphates or glycinates of sodium, potassium or ammonium, or mixtures thereof.

11. The formulation according to claim 1, further comprising at least one sweetener.

12. The formulation according to claim 11, wherein said at least one or more sweetener is selected from the group consisting of sucrose, aspartame, acesulfame potassium, saccharine, sodium saccharine, cyclamate, glycyrrhizine, thaumatin, sucralose, neohesperidin dihydrochalcone, alitame, miraculin, monellin, stevside, salts and mixtures thereof.

13. The formulation according to claim 1, further comprising at least one emulsifier.

14. The formulation according to claim 1, wherein said at least one emulsifier is selected from the group consisting of
lecithin, poloxamer, polyoxyethylene alkyl ether, polyoxyethylene castor oil derivative, polyoxyethylene sorbitan fatty acid ester, monoglyceride, diglyceride and esther thereof, polyoxyethylene stearate, polyglycerolester of fatty acids, sorbitan fatty acid ester, fatty acid, soap of fatty acid, lactylate, sodium lauryl sulfate, latanol, phosphatidylcholine, phosphatidylethanolamine, and mixtures thereof.

15. The formulation according to claim 14, wherein said emulsifier is lecithin.

16. The formulation according to claim 1, further comprising a substance selected from the group consisting of sucrose, fructose, glucose, galactose, lactose, maltose, invert sugar, xylitol, sorbitol, maltitol, mannitol, isomalt, glycerol, polydextrose, starch, or mixtures thereof.

17. The formulation according claim 1, further comprising one or more other agents having an effect against overactive bladder.

18. The formulation according to claim 17, wherein said one or more agents is selected from the group consisting of oxobutynin, emepromium, trospium, propanetheline, darifenacin and mixtures thereof.

19. A method for treating overactive bladder in a subject comprising administration of a tolterodine-containing orally administered pharmaceutical formulation according to claim 1.
